(19) United States
(12) Patent Application Publication
(10) Pub. No.: US 2005/0278870 A1
(43) Pub. Date: Dec. 22, 2005

(54) Method for providing custom hair colour products

(76) Inventor: Alfredo Gaspini, Toronto (CA)

Correspondence Address: GROSSMAN & FLIGHT LLC, Suite 4220, 20 North Wacker Drive, Chicago, IL, 60606-6357, US

(21) Appl. No.: 10/873,043

(22) Filed: Jun. 22, 2004

(51) Int. Cl. A61K 8/00

(52) U.S. Cl. 8/405

(57) ABSTRACT

A method of providing a consumer with a custom hair colouring product. Details of personal characteristics are obtained from the consumer with the assistance of a licensed hair colouring technician, including a desired hair colour. A custom formula is developed based upon the personal characteristics of the consumer, wherein the formula prescribes two or more base dye colours and their relative quantities. The consumer is provided with the two more base dye colours and a developer agent and self-applies the custom hair colouring product.

### FIELD OF THE INVENTION

The present invention relates to a method for providing custom hair colour products and, in particular, a method for customizing hair colour products to an individual consumer in a retail environment.

### BACKGROUND OF THE INVENTION

Consumers currently have two options available to them when it comes to obtaining hair colouring. First, there are commercially available pre-packaged hair colouring products marketed by companies such as Revlon Consumer Products Corporation and L'Oreal Paris. These products are typically marketed through drug stores and other retail outlets. They usually constitute a two-component kit having a particular dye mixture in one container and a developer composition in another container. Once the consumer has purchased the product and returned home, the consumer combines the dye mixture and developer composition and applies the combination to his or her hair. To choose his or her hair colour, the consumer selects from the available shades offered by the retail outlet. A typical drug store outlet may allocate approximately 15 feet of shelving space to hair colouring products, often providing a selection of brands. Each brand may have a couple dozen shades available, although not all shades may be available at every retail outlet. A particular shade of commercially available hair colouring product will produce different results for consumers having different personal characteristics. Accordingly, a great deal of experimentation is required on the behalf of the consumer to discover a brand and a shade that provides the appropriate result. Many consumers never obtain a satisfactory result from pre-packaged hair colouring products.

The other option is to have hair colouring performed by a licensed hair stylist in a salon. In this case, the hair stylist will select a dye or combination of dyes from a wide range of base dye colours. The selection of particular dyes is based upon the desired hair colour and the individual characteristics of the consumer's hair. The stylist then mixes together the appropriate base dyes and a developer and applies the mixture in the salon. Accordingly, the consumer receives a hair colouring treatment that uses a custom dye mixture tailored to his or her individual characteristics.

Hair colouring through a salon is many times more expensive than the commercially available pre-packaged hair colouring products. However, many consumers require the expert and individual advice provided by the hair stylist. The selection of a custom dye mixture for the individual consumer is more likely to produce a satisfactory colour result. Moreover, the wide variety of base dyes available to the hair stylist means that there are far more colour combinations possible than are available in pre-packaged hair colour products. The blend formula chosen for each individual is a carefully guarded secret of the hair stylist. Typically, the stylist does not share the formula with the consumer since the consumer could take the formula to another salon for his or her next colouring.

It would be desirable to provide consumers with the advantages of custom hair colouring without the associated expense of an in-salon hair colouring.

### SUMMARY OF THE INVENTION

The present invention relates to a method for providing a consumer with a custom hair colouring product. The method may be practiced through a retail outlet or kiosk or the like. A blend formula identifying two or more base dyes is determined based upon consumer input. The two or more base dyes are provided to the consumer in a blended or unblended form along with a developer. The consumer then self-applies the custom formulated blend of dyes, for example at his or her residence, to colour his or her hair. A licensed colour technician may determine one or more of the personal characteristics used in determining the formula.

In one aspect, the present invention provides a method for providing custom hair colour products to a consumer in a retail outlet. The method includes the steps of obtaining input from the consumer, including at least one personal characteristic and a desired hair colour, determining a formula based upon the input, the formula identifying at least two base dyes and their relative quantities, and providing the consumer with the at least two base dyes and a developer. The formula is determined so as to provide the consumer with the desired hair colour once the consumer applies the at least two base dyes and the developer.

In another aspect the present invention provides a method of obtaining custom hair colour products from a retail outlet. The method includes the steps of providing input, including at least one personal characteristic and a desired hair colour, receiving at least two base dye colours and a developer composition, the at least two base dye colours being prescribed by a custom formula determined based upon the input, and wherein the custom formula is determined so as to provide the desired hair colour, and self-applying the at least two base dye colours and the developer composition.

Other aspects and features of the present invention will be apparent to those of ordinary skill in the art from a review of the following detailed description when considered in conjunction with the drawings.

### BRIEF DESCRIPTION OF THE DRAWINGS

Reference will now be made, by way of example, to the accompanying drawings which show an embodiment of the present invention, and in which:

FIG. 1 shows, in flowchart form, an embodiment of a method of providing a consumer with a custom hair colouring product in accordance with the present invention; and

FIG. 2 shows, in flowchart form, another embodiment of a method of providing a consumer with a custom hair colouring product in accordance with the present invention.

Similar reference numerals are used in different figures to denote similar components.

### DESCRIPTION OF SPECIFIC EMBODIMENTS

Although the following description may make reference to products for colouring hair, it will be understood by those of ordinary skill in the art that the present invention is not limited to permanent hair colouring products alone and that the term “hair colouring products” includes permanent, semi-permanent, and temporary colouring products, and includes products for highlighting, streaking, and lightening. It will also be understood that any references herein to specific dyes, developer agents, or compositions should not be understood as a limitation of the present invention.

The present invention contemplates providing a consumer with a custom hair colouring product that the consumer applies his or herself. The custom hair colouring product is based upon a formula determined to provide the specific consumer with a desired hair colour. The formula specifies two or more base dye colours and their relative quantities. When blended in the specified quantities, the base dye colours produce a custom blended dye colour.

The formula is developed to achieve the desired hair colour while taking into account the personal characteristics of the individual consumer. In this manner the present invention captures the advantages of in-salon hair colouring. The present invention also provides the cost and flexibility advantages of home self-application.

In practice, the consumer approaches a retail outlet, such as a store-front location or kiosk. The retail outlet may be located in a permanent shopping mall or may be temporarily located at special events or other locations.

The retail outlet may be staffed by at least one licensed colour technician. The licensed colour technician is a person having sufficient training to identify and specify personal characteristics associated with hair colouring. For example, a licensed colour technician may examine a consumer and identify the porosity of the consumer's hair or identify the greyness of a consumer's hair. These are personal characteristics that consumers are usually incapable of accurately specifying.

The retail outlet is stocked with a selection of base dye colours. The inventory of base dye colours may, in one embodiment, comprise individual base dye colours in one ounce tubes or containers for retail distribution. In one embodiment, the retail outlet includes at least fifty base dye colours. It will be understood that the base dye colours may be stored in other types or sizes of containers.

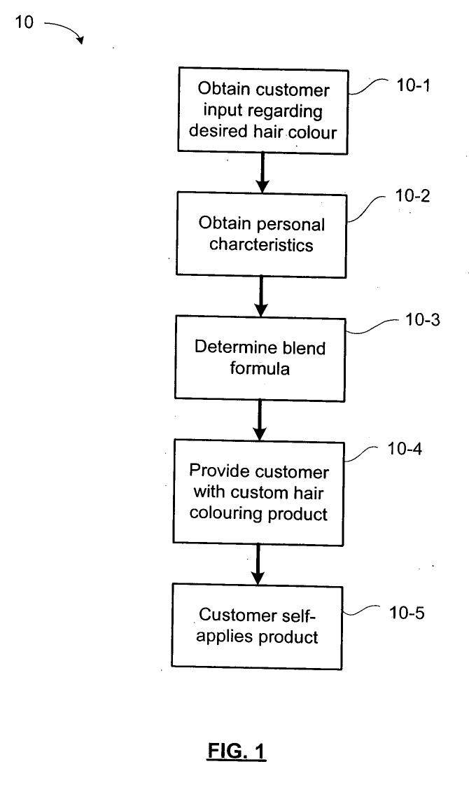

Reference is now made to FIG. 1, which shows, in flowchart form, an embodiment of a method 10 of providing a consumer with a custom hair colouring product in accordance with the present invention.

On entering the retail outlet, the consumer may meet with the licensed colour technician. The first step in determining an appropriate colour formula is to obtain input from the consumer, as indicated by steps 10-1 and 10-2. Input may be obtained from the consumer in a variety of ways. The licensed colour technician may question the consumer, for example regarding the consumer's desired hair colour. The licensed colour technician may present the consumer with a menu of options, for example sample photographs or depictions of possible desired hair colours. Alternatively, the consumer may be presented with a questionnaire, in written or electronic form, through which the consumer provides the input. An electronic questionnaire may be provided through a touch-screen terminal allowing the consumer to select options, such as a desired hair colour. These various methods of obtaining input from the consumer may be combined to obtain the most accurate input. In particular, certain input, such as the consumer's hair porosity or greyness levels may be difficult for the consumer to accurately gauge and may require examination and assessment by the licensed hair colour technician. In the case of an electronic or written form, the licensed hair colour technician may provide the consumer with guidance regarding input of this nature.

The consumer may be prompted to provide a number of different inputs, including desired hair colour, base hair colour, ethnicity, hair porosity, and greyness levels. Those of ordinary skill in the art will appreciate that other input may be solicited to further refine the ability to accurately formulate a colour blend to provide the consumer with the desired hair colour. Other input may include details regarding any residual colouring in the consumer's hair, desired highlight details, skin colouring, and/or desired colour tone, e.g. warm, ash, etc.,

Input obtained from the consumer may also include non-hair related personal information to be used in creating a consumer entry in a consumer database. The consumer entry in the consumer database may be used to track the consumer's hair colouring over time and to store details regarding past formulas developed for the consumer.

In step 10-3, an appropriate dye formula is determined based upon the consumer input, including the details of the consumer's hair and the desired hair colour. The details regarding the consumer's hair, including the base hair colour, the porosity, and the greyness level, determine the necessary formula for providing the consumer with the desired hair colour. The appropriate custom formula for the consumer may be determined by the licensed hair colouring technician, taking into account the consumer input. Alternatively, the appropriate custom formula may be determined using a computer system configured to calculate the custom formula in response to the consumer input. The computer system for determining the appropriate custom formula may be integrated with the terminal used to solicit input from the consumer, with assistance from the licensed hair colour technician. The computer system may be employed as a check to verify that the colour technician's determination of an appropriate formula falls within a generally accepted range of solutions.

The custom formula specifies two or more base dye colours which, when blended in the prescribed quantities, produce a custom colour blend specifically formulated to provide the individual consumer with his or her desired hair colour. The formula further specifies the relative quantities of the base dye colours to be blended to obtain the custom colour blend.

In general, a custom formula may take the form:

$$\frac{x}{2} [ {a}_{1} {D}_{1} + {a}_{2} {D}_{2} + … + {a}_{n} {D}_{n} ] + \frac{x}{2} H$$

where Dₙ is a dye lot/colour, H is a developer composition, x is the quantity of the overall mixture in, for example, fluid ounces or milliliters, and aₙ is the relative quantity of dye n as compared to other dyes and provided that:

$${\sum }_{i = 1}^{n} {a}_{i} = 1$$

An example of a custom formula is based upon a consumer having natural level four hair with 75% grey content and desiring a chestnut brown colour. The natural level of a consumer's hair is based upon the relative lightness or darkness of the hair pigment and is not related to the colour. Natural level is typically scored on a scale of one to ten, with one being the darkest blackest level and ten being the lightest whitest level. The custom formula developed for such an example customer may, for example, be:
50 mL (0.33 dye1+0.33 dye2+0.33 dye3)+50 mL (10 vol. H₂O₂)
where dye 1 may be dye number 700 for grey coverage, and dyes 2 and 3 may be dye numbers 55 and 657, respectively, for golden appearance. The example dye numbers refer to dye numbers associated with dyes marketed by Schwarzkopf & Henkel of Germany. It will be understood that different types of dyes from different sources may be used.

Once the custom formula is determined, then in step 10-4 the licensed colour technician provides the consumer with the two or more base dye colours and the formula prescribing the relative quantities in which the base dye colours are to be blended. In one embodiment, the licensed colour technician provides the consumer with each base dye colour in a separate container. Each base dye colour may be provided in predetermined quantities, e.g. in one-ounce tubes or vials, in which case the consumer may receive more than one container of each dye colour. Alternatively, the dye colours may be packaged in separate containers but in custom quantities.

Along with the formula and the base dye colours, the consumer is provided with an activating agent, such as hydrogen peroxide or other bleaches. The activating agent comprises a developer composition for combination with the dyes when applying the custom hair colouring product.

The consumer leaves the retail outlet with the custom hair colour product, i.e. the dyes and the developer composition. In step 10-5, the consumer applies the custom hair colour product. In particular, at an appropriate location including, but not limited to, a hotel room or the consumer's personal residence, the consumer blends the base dye colours in the relative quantities prescribed by the formula to produce a custom colour blend. The consumer then combines the custom colour blend and the activating agent or developer composition and applies the mixture to his or her hair. The custom hair colour product may further include instructions to guide the consumer regarding the blending, mixing, and application of the custom hair colouring product. The instructions may include guidance regarding how to apply the product, how long to leave the product on, and how to rinse or remove the product.

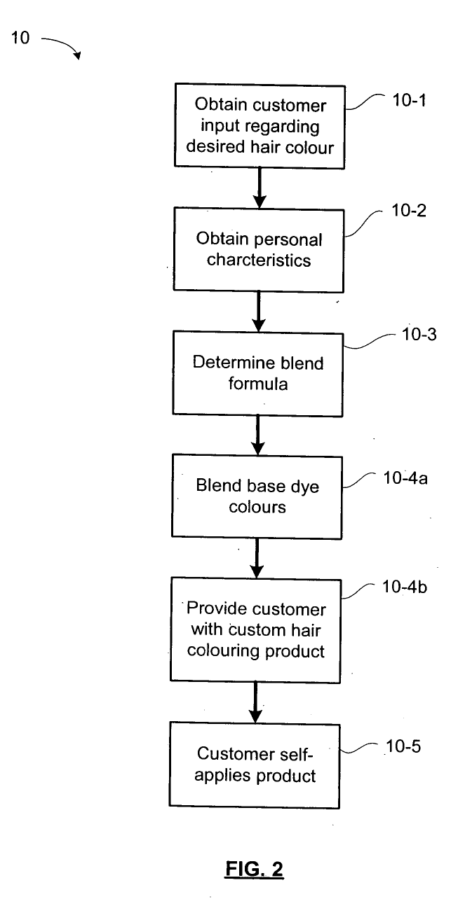

Reference is now made to FIG. 2, which shows, in flowchart form, an alternative embodiment of the method 10 shown in FIG. 1. In this embodiment, the licensed hair colouring technician blends the base dye colours in accordance with the formula to produce the custom blended colour, as indicated by step 10-4a. In step 10-4b, the consumer is provided with the custom blended colour and with the activating agent in separate containers. The consumer may also be provided with the custom formula, although in this embodiment the consumer is not required to blend the colour his or herself. Accordingly, in step 10-5, the consumer mixes the blended custom colour and the activating agent and applies the mixture in accordance with the application instructions.

Advantageously, the present invention provides a consumer with a self-applied custom formulated hair colouring product specifically created to provide the consumer with his or her desired hair colour having regard to his or her personal characteristics.

Once a consumer's custom personal formula has been determined, the consumer may re-order the custom hair colour product, in person, on-line, by telephone, or through any other retail exchange. The consumer can also tweak the formula to slightly alter the effects. For example, the consumer may wish to try a colouring having slightly more red tint, in which case the previously determined custom formula stored and associated with the consumer's records may be modified to provide for the additional red tint. Subtle variations may also be specified through an on-line or telephone order without requiring a further visit to the retail outlet.

The present invention may be embodied in other specific forms without departing from the spirit or essential characteristics thereof. Certain adaptations and modifications of the invention will be obvious to those skilled in the art. Therefore, the above discussed embodiments are considered to be illustrative and not restrictive, the scope of the invention being indicated by the appended claims rather than the foregoing description, and all changes which come within the meaning and range of equivalency of the claims are therefore intended to be embraced therein.

1. A method for providing custom hair colour products to a consumer in a retail outlet, comprising the steps of:
(a) obtaining input from the consumer, including at least one personal characteristic and a desired hair colour;
(b) determining a formula based upon said input, said formula identifying at least two base dyes and their relative quantities; and
(c) providing the consumer with said at least two base dyes and a developer,
whereby said formula is determined so as to provide the consumer with said desired hair colour once the consumer applies the at least two base dyes and the developer.

2. The method claimed in claim 1, wherein said step of providing includes providing the consumer with said at least two base dyes in an unmixed form.

3. The method claimed in claim 2, wherein said at least two base dyes are provided to the consumer in separate containers.

4. The method claimed in claim 2, wherein said step of providing further includes providing the consumer with said formula for blending said at least two base dyes.

5. The method claimed in claim 1, further including a step of blending said at least two base dyes to produce a blended dye, and wherein said step of providing comprises providing the consumer with said blended dye and said developer.

6. The method claimed in claim 1, wherein said step of determining includes selecting said at least two base dyes from among at least fifty base dyes.

7. The method claimed in claim 1, further including a step of providing the consumer with said formula.

8. The method claimed in claim 7, further including a step of providing the consumer with instructions regarding the self-application of said at least two dyes and said developer.

9. The method claimed in claim 8, wherein said instructions further include instructions regarding the blending of said at least two dyes.

10. The method claimed in claim 1, further including a step of storing said formula in a database in association with a consumer identity.

11. The method claimed in claim 1, wherein said step of obtaining input from the consumer includes examining the consumer and determining said at least one personal characteristic.

12. The method claimed in claim 11, wherein said steps of examining the consumer and determining said at least one personal characteristic are performed by a licensed colour technician.

13. The method claimed in claim 1, wherein said step of determining said formula is performed by a licensed colour technician.

14. The method claimed in claim 1, wherein said step of determining said formula is performed by a computer system.

15. The method claimed in claim 1, wherein said at least one personal characteristic includes a characteristic selected from the group consisting of ethnicity, base hair colour, hair porosity, and greyness.

16. The method claimed in claim 1, wherein said at least one personal characteristic comprises ethnicity, base hair colour, hair porosity, and greyness.

17. The method claimed in claim 1, wherein said at least two base dyes and said developer are provided in separate containers.

18. A method of obtaining custom hair colour products from a retail outlet, comprising the steps of:
(a) providing input, including at least one personal characteristic and a desired hair colour;
(b) receiving at least two base dye colours and a developer composition, said at least two base dye colours being prescribed by a custom formula determined based upon said input, and wherein said custom formula is determined so as to provide said desired hair colour; and
(c) self-applying said at least two base dye colours and said developer composition.

19. The method claimed in claim 18, wherein said step of receiving said at least two base dye colours includes receiving said at least two base dye colours in an unmixed form in separate containers, and wherein said method further includes a step of mixing said at least two base dye colours in accordance with said custom formula prior to said step of self-applying.

20. A custom hair colouring product produced in accordance with the method claimed in claim 1.
